(19) United States
(12) Patent Application Publication
(10) Pub. No.: US 2026/0365614 A1
(43) Pub. Date: Nov. 19, 2026

(54) CARBON-BASED POLY(ESTER-URETHANE-UREA) MICROCAPSULES

(71) Applicant: FMC CORPORATION, Philadelphia, PA (US)

(72) Inventors: Sergiy PELESHANKO, Hockessin, DE (US); Maura HENDRIXSON, Newark, DE (US)

(21) Appl. No.: 19/663,710

(22) Filed: Apr. 30, 2026

Related U.S. Application Data
(60) Provisional application No. 63/797,890, filed on Apr. 30, 2025.

(51) Int. Cl. C07C 265/08

(52) U.S. Cl. CPC C07C 265/08

(57) ABSTRACT

This invention is directed to the reaction product of a tri-ol compound of Formula 1,

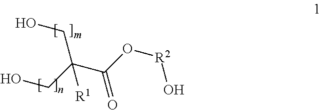

wherein m, n, R¹ and R² are as defined in the Summary of the Invention and at least one starter di-isocyanate compound.

More particularly, this invention relates to the reaction product of a tri-ol compound of Formula 1 and at least one starter di-isocyanate compound as defined and described above in the Summary of the Invention. This Invention also relates to micrcopsules prepared from the reaction product of a tri-ol compound of Formula 1 as described in the Summary of the Invention and a diamine compound. This Invention also relates to a formulation (i.e. composition) containing the microcapsules prepared from the reaction product described in the Summary of the Invention, further comprising surfactants, solid diluents and liquid diluents.

### FIELD OF THE INVENTION

This invention relates to certain microcapsule precursors as pre-polymer compounds, their stereoisomers, and methods of their use for preparing microcapsules.

### BACKGROUND OF THE INVENTION

Encapsulation of agrochemical active ingredients aids in releasing the agrochemical active ingredients in a controlled manner, extending the activity of the agrochemical active ingredients for extended durations, protecting the agrochemical active ingredients from UV radiation, controlling the evaporation of volatile agrochemical active ingredients, and/or avoiding crop injury. Thus, encapsulation of agrochemical active ingredients is beneficial and desirable for numerous reasons.

Polyurea microcapsules produced by interfacial polymerization of isocyanates and amines are widely used to encapsulate agrochemicals. However, current polyurea microcapsules do not undergo sufficient degradation in the environment. As a result, the widespread use of microplastics like polyurea microcapsules has polluted the marine environment and bans of microplastics are contemplated by regulatory agencies. Accordingly, there is a need for new microcapsules suitable for use with agrochemicals.

WO2024/158782 discloses a biodegradable core-shell microcapsule slurry composed of microcapsules. The polyisocyanate compounds of the present invention are not disclosed in this reference. Ang. Chem. Int. Ed. 2015 54, 2275-2279 describes certain urea-based microcapsules for encapsulating biologically active ingredients. The microcapsules of the present invention are not disclosed in this reference.

### SUMMARY OF THE INVENTION

This invention is directed to the reaction product of a tri-ol compound of Formula 1, stereoisomers thereof,

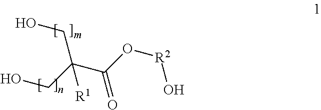

wherein
m is an integer from 1 to 4;
n is an integer from 1 to 4;
R¹ is selected from H, unsubstituted phenyl and linear and branched C₁-C₆ alkyl;
R² is selected from linear and branched C₂-C₆ alkyl and saturated or unsaturated
   C₅-C₆ cycloalkyl, optionally substituted with a linear or branched C₁-C₆ alkyl and at least one starter di-isocyanate compound.

More particularly, this invention relates to the reaction product of a tri-ol compound of Formula 1 and at least one starter di-isocyanate compound as defined and described above in the Summary of the Invention. This Invention also relates to micrcopsules prepared from the reaction product of a tri-ol compound of Formula 1 as described in the Summary of the Invention and a diamine compound. This Invention also relates to a formulation (i.e. composition) containing the microcapsules prepared from the reaction product described in the Summary of the Invention, further comprising surfactants, solid diluents and liquid diluents.

### DETAILS OF THE INVENTION

It was discovered herein that carbon-based polyurea-based microcapsules encapsulating agrochemical active ingredients could be made by including at least one ester group in the shell of the microcapsules. The modified microcapsules are carbon-based poly(ester-urethane-ureas) (PEUUs). The microcapsules of the present disclosure may be used to encapsulate a wide variety of agrochemical active ingredients.

In one embodiment, the present disclosure is directed to a microcapsule encapsulating an agrochemical active ingredient comprising the reaction product of a compound of Formula 1 and at least one starter di-isocyanate compound and at least one amine-containing compound, wherein the at least one isocyanate-containing compound and/or the at least one amine-containing compound comprises at least one ester group, and wherein the reaction product comprises at least one ester group.

In another embodiment, the present disclosure is directed to a method of making a microcapsule, the microcapsule comprising the reaction product of the compound of Formula 1, at least one starter di-isocyanate compound and at least one amine-containing compound, wherein the at least one isocyanate-containing compound and/or the at least one amine-containing compound comprises at least one ester group, and wherein the reaction product comprises at least one ester group, wherein the method comprises encapsulating the core with the shell.

In yet another embodiment, the present disclosure is directed to a method for controlling a pest, wherein the method comprises applying to the pest or an environment of the pest a composition comprising a microcapsule comprising the reaction product of a compound of Formula 1 and a starter di-isocyanate compound; and at least one amine-containing compound, microencapsulating an agrochemical active ingredient containing at least one is applied as a formulated material wherein the at least one, and wherein the reaction product comprises at least one ester group.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a compound, composition, mixture, process or method that comprises a list of elements is not necessarily limited to only those elements but may include other elements not expressly listed or inherent to such compound, composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “of” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Cycloalkyl” includes, for example, cyclopropyl, cyclobutyl, cyclopentyl and cyclohexyl.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers.

Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial microencapsulation efficiency when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

The tri-ol compound of Formula 1 possess a chiral center. The enantiomers are depicted as Formula 1′ and Formula 1″ (i.e. the tri-ol compound of Formula 1 when m is 2 and n is 1 as defined in the Summary of the Invention) with the chiral center identified with an asterisk (*). For a comprehensive discussion of all aspects of stereoisomerism, see Ernest L. Eliel and Samuel H. Wilen, Stereochemistry of Organic Compounds, John Wiley & Sons, 1994.

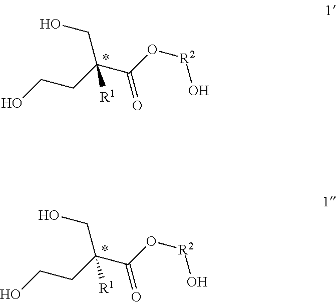

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer.

Constant width lines indicate bonds with a direction opposite or neutral relative to bonds shown with solid or dashed wedges; constant width lines also depict bonds in molecules or parts of molecules in which no particular stereoconfiguration is intended to be specified.

Preferably, this invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x−1) 100%, where x is the mole fraction of the dominant enantiomer in the mixture (e.g., an ee of 20% corresponds to a 60:40 ratio of enantiomers). The pre-polymer compounds of this invention can have at least a 50% enantiomeric excess; more preferably at least a 75% enantiomeric excess; still more preferably at least a 90% enantiomeric excess; and the most preferably at least a 94% enantiomeric excess of the more active isomer.

Compounds of Formula 1 can comprise additional chiral centers. For example, substituents and other molecular constituents such as R¹ and R² may themselves contain chiral centers. This invention comprises racemic mixtures as well as enriched and essentially pure stereoconfigurations at these additional chiral centers.

Compounds of this invention can exist as one or more conformational isomers due to restricted rotation about any amide bonds present (e.g., in M) in Formula 1. This invention comprises mixtures of conformational isomers. In addition, this invention includes compounds that are enriched in one conformer relative to others.

Compounds of Formula 1 can exist in more than one form, and Formula 1 thus also include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

This invention relates to a pre-polymer compound of Formula 1 as defined and described above in the Summary of the Invention. This invention also relates to a method for preparing a microencapsulated formulation from the compound of Formula 1. This invention also relates to a polymerized microencapsulated formulation incorporating an agrochemical. This invention is further defined as described in any of the Embodiments below.

Embodiment 1. The reaction product of a compound of Formula 1, stereoisomers thereof and at least one di-isocyanate compound as defined and described in the Summary of the Invention.

Embodiment 2. The reaction product of Embodiment 1 wherein the compound of Formula 1, stereoisomers thereof wherein m is 1 to 3.

Embodiment 3. The compound of Embodiment 2 wherein m is 1 to 2.

Embodiment 4. The compound of Embodiment 3 wherein m is 1.

Embodiment 5. The reaction product of any one of Embodiments 1 through 4 wherein the compound of Formula 1, stereoisomers thereof wherein n is 1 to 3.

Embodiment 6. The compound of Embodiment 5 wherein n is 1 to 2.

Embodiment 7. The compound of Embodiment 6 wherein n is 1.

Embodiment 8. The reaction product of any one of Embodiments 1 through 7 wherein the compound of Formula 1, stereoisomers thereof wherein R¹ is selected from H and linear C₁-C₆ alkyl.

Embodiment 9. The compound of Embodiments 8 wherein R¹ is selected from H, —CH₃, —CH₂CH₃ and —CH₂CH₂CH₃.

Embodiment 10. The compound of Embodiment 9 wherein R¹ is selected from H, —CH₃ and —CH₂CH₃.

Embodiment 11. The reaction product of any one of Embodiments 1 through 10 wherein the compound of Formula 1, stereoisomers thereof wherein R² is selected from linear and branched C₂-C₆ alkyl and saturated or unsaturated C₅ cycloalkyl, optionally substituted with a linear C₁-C₆ alkyl.

Embodiment 12. The compound of Embodiment 11 wherein R² is selected from linear C₂-C₆ alkyl and saturated C₅ cycloalkyl, optionally substituted with a linear C₁-C₃ alkyl.

Embodiment 13. The compound of Embodiment 12 wherein R² is selected from linear C₂-C₄ alkyl and saturated C₅ cycloalkyl, optionally substituted with CH₃.

Embodiment 14. The compound of Embodiment 13 wherein R² is selected from linear C₂-C₄ alkyl and unsubstituted saturated C₅ cycloalkyl.

Embodiment 15. The compound of Embodiment 14 wherein R² is selected from linear C₂-C₃ alkyl.

Embodiment 16. The compound of Embodiment 15 wherein R² is selected from linear C₂ alkyl.

Embodiment 17. The compound of Embodiment 15 wherein R² is selected from linear C₃ alkyl.

Embodiment 18. The reaction product as described in the Summary of the Invention and any one of Embodiments 1 through 17 wherein the at least one starter di-isocyanate compound is aliphatic, cycloaliphatic, or aromatic.

Embodiment 19. The reaction product described in Embodiment 18, wherein the at least one starter di-isocyanate compound is aliphatic.

Embodiment 20. The reaction product described in Embodiment 18, wherein the at least one starter di-isocyanate compound is cycloaliphatic.

Embodiment 21. The reaction product described in Embodiment 18, wherein the at least one starter di-isocyanate containing compound is aromatic.

Embodiment 22. The reaction product described in any of Embodiments 1 through 21 wherein the at least one starter di-isocyanate compound is selected from 1,6-hexamethylene diisocyanate (HDI), 2,2,4- and 2,4,4-trimethyl-1,6-hexamethylene diisocyanate (TMDI), 1-isocyanato-3,3,5-trimethyl-5-isocyanatomethylcyclohexane (isophorone diisocyanate or IPDI), perhydro-2,4′- and -4,4′-diphenylmethane diisocyanate (HMDI), 1,4-diisocyanato-2,2,6-trimethylcyclohexane (TMCDI), 1,3- and 1,4-bis(isocyanato methyl)cyclohexane, m-xylylene diisocyanate (m-XDI), p-xylylene diisocyanate (p-XDI), m- and p-tetramethyl-1,3- and -1,4-xylylene diisocyanate (m- and p-TMXDI), 2,4- and 2,6-tolylenediisocyanate (TDI), 4,4′-2,4′- and 2,2′-diphenylmethane diisocyanate (MDI), polymeric MDI (PMDI), homologues thereof, isomers thereof, and mixtures thereof.

Embodiment 23. The reaction product described in Embodiment 22 wherein the at least one starter di-isocyanate compound comprises 1,6-hexamethylene diisocyanate (HDI).

Embodiment 24. The reaction product described in Embodiment 22 wherein the at least one starter di-isocyanate compound comprises 2,2,4- and/or 2,4,4-trimethyl-1,6-hexamethylene diisocyanate (TMDI).

Embodiment 25. The reaction product described in Embodiment 22, wherein the at least one starter di-isocyanate compound comprises 1-isocyanato-3,3,5-trimethyl-5-isocyanatomethylcyclohexane (isophorone diisocyanate or IPDI).

Embodiment 26. The reaction product described in Embodiment 22, wherein the at least one starter di-isocyanate compound comprises perhydro-2,4′- and/or -4,4′-diphenylmethane diisocyanate (HMDI).

Embodiment 27. The reaction product described in Embodiment 22, wherein the at least one starter di-isocyanate compound comprises 1,4-diisocyanato-2,2,6-trimethylcyclohexane (TMCDI).

Embodiment 28. The reaction product described in Embodiment 22, wherein the at least one starter di-isocyanate compound comprises 1,3- and/or 1,4-bis(isocyanato methyl)cyclohexane.

Embodiment 29. The reaction product described in Embodiment 22, wherein the at least one starter di-isocyanate compound comprises m-xylylene diisocyanate (m-XDI).

Embodiment 30. The reaction product described in Embodiment 22, wherein the at least one starter di-isocyanate compound comprises p-xylylene diisocyanate (p-XDI).

Embodiment 31. The reaction product described in Embodiment 22, wherein the at least one starter di-isocyanate compound comprises m- and/or p-tetramethyl-1,3- and/or -1,4-xylylene diisocyanate (m- and p-TMXDI).

Embodiment 32. The reaction product described in Embodiment 22, wherein the at least one starter di-isocyanate compound comprises 2,4- and/or 2,6-tolylenediisocyanate (TDI).

Embodiment 33. The reaction product described in Embodiment 22, wherein the at least one starter di-isocyanate compound comprises 4,4′- and/or 2,4′- and/or 2,2′-diphenylmethane diisocyanate (MDI).

Embodiment 34. The reaction product described in Embodiment 22, wherein the at least one starter di-isocyanate compound comprises polymeric MDI (PMDI).

Embodiment 35. The reaction product described in Embodiment 22, wherein the at least one starter di-isocyanate compound comprises a homologue of the at least one starter di-isocyanate compound.

Embodiment 36. The reaction product described in Embodiment 22, wherein the at least one starter di-isocyanate compound comprises an isomer of the at least one di-isocyanate compound.

Embodiment 37. A polymer microcapsule prepared from the reaction product of any one of Embodiments 1 though 36 and an amine selected from a di-amine or a tri-amine.

Embodiment 38. The polymer microcapsule described in Embodiment 37 wherein the di-amine or tri-amine comprises an aliphatic di-amine or an aliphatic tri-amine.

Embodiment 39. The polymer microcapsule described in Embodiment 38 wherein the di-amine or tri-amine is selected from polyester di-amines, polyester tri-amines, oligoesteramines, ester diamines, diester diamines, polyester polyols comprising at least two amino groups, and combinations thereof.

Embodiment 40. The polymer microcapsule described in Embodiment 39, wherein the di-amine or tri-amine compound comprises a polyester amine.

Embodiment 41. The polymer microcapsule described in Embodiment 40, wherein the di-amine or tri-amine compound comprises a polyester di-amine or polyester tri-amine.

Embodiment 42. The microcapsule described in Embodiment 41, wherein the di-amine or tri-amine compound comprises an oligoesteramine.

Embodiment 43. The polymer microcapsule described in Embodiment 42, wherein the di-amine or tri-amine compound comprises an ester diamine.

Embodiment 44. The polymer microcapsule described in Embodiment 43, wherein the di-amine or tri-amine compound comprises a diester diamine.

Embodiment 45. The polymer microcapsule as defined in Embodiment 37 wherein the amine is a di-amine.

Embodiment 46. The polymer microcapsule of Embodiment 45 wherein the di-amine is selected from the group consisting of ethylenediamine (EDA), 1,2-propanediamine, 1,3-propanediamine (DAP), 2-methyl-1,2-propanediamine, 2,2-dimethyl-1,3-propanediamine, 1,3-butanediamine, 1,4-butanediamine, 1,3-pentanediamine, 1,5-pentanediamine, 2-butyl-2-ethyl-1,5-pentanediamine, 1,6-hexamethylenediamine (HMDA), methylbis(3-aminopropyl)amine, 1,5-diamino-2-methylpentane (MPMD), 1,2-diaminocyclohexane, 1,3-diaminocyclohexane, 1,4-diaminocyclohexane, bis(4-aminocyclohexyl)methane, bis(4-amino-3-methylcyclohexyl)methane, 1-amino-3-aminomethyl-3,5,5-trimethylcyclohexane (isophoronediamine or IPDA), m-xylylenediamine (XDA), 1,3-bis(aminomethyl)cyclohexane (BAC), methylene-bis(cyclohexylamine) (MBCHA), 4,4′-methylenebis(2-methylcyclohexylamine) (MBMCHA), homologues thereof, isomers thereof, and combinations thereof.

Embodiment 47. The polymer microcapsule described in Embodiment 46 wherein the di-amine compound comprises ethylenediamine (EDA).

Embodiment 48. The polymer microcapsule described in Embodiment 46 wherein the di-amine compound comprises 1,3-propanediamine (DAP).

Embodiment 49. The polymer microcapsule described in Embodiment 46 wherein the di-amine compound comprises 2,2-dimethyl-1,3-propanediamine.

Embodiment 50. The polymer microcapsule described in Embodiment 46 wherein the di-amine compound comprises 1,6-hexamethylenediamine (HMDA).

Embodiment 51. The polymer microcapsule described in Embodiment 46 wherein the di-amine compound comprises 1,2-diaminocyclohexane.

Embodiment 52. The polymer microcapsule described in Embodiment 46 wherein the di-amine compound comprises bis(4-aminocyclohexyl)methane.

Embodiment 53. The polymer microcapsule described in Embodiment 46 wherein the di-amine compound comprises bis(4-amino-3-methylcyclohexyl)methane.

Embodiment 54. The polymer microcapsule described in Embodiment 46 wherein the di-amine compound comprises m-xylylenediamine (XDA).

Embodiment 55. The polymer microcapsule described in Embodiment 46, wherein the di-amine compound comprises 1,3-bis(aminomethyl)cyclohexane (BAC).

Embodiment 56. The polymer microcapsule described in Embodiment 46, wherein the di-amine compound comprises methylene-bis(cyclohexylamine) (MBCHA).

Embodiment 57. The polymer microcapsule described in Embodiment 46, wherein the di-amine compound comprises 4,4′-methylenebis(2-methylcyclohexylamine) (MBMCHA).

Embodiment 58. The polymer microcapsule as defined in Embodiment 37 wherein the amine is a tri-amine.

Embodiment 59. The polymer microcapsule as defined in Embodiment 58 wherein the triamine is 1,3,6 tri-aminohexane.

Embodiment 60. A polymer microcapsule prepared as defined in any one of Embodiments 37 through 59 from the reaction product as defined in any one of Embodiments 1 through 36 wherein the polymer microcapsule exhibits a microencapsulation efficiency of greater than 95%.

Embodiment 61. The polymer microcapsule of Embodiment 60 wherein the polymer microcapsule exhibits a microencapsulation efficiency of greater than 96%.

Embodiment 62. A polymer microcapsule prepared as defined in any one of Embodiments 37 through 59 from the reaction product as defined in any one of Embodiments 1 through 36 wherein the polymer microcapsule has a particle size D90 of less than 50 microns (micrometers).

Embodiment 63. The polymer microcapsule as defined in Embodiment 62 wherein the polymer microcapsule has a particle size D90 of less than 40 microns.

Embodiment 64. The polymer microcapsule as defined in Embodiment 63 wherein the polymer microcapsule has a particle size D90 of between 5 and 8 microns.

Embodiment 65. The polymer microcapsule as defined in Embodiment 63 wherein the polymer microcapsule encapsulating a pheromone has a particle size D90 of less than 30 microns.

Embodiment 66. The polymer microcapsule as defined in Embodiment 63 wherein the polymer microcapsule encapsulating a herbicide has a particle size D90 of less than 15 microns.

Embodiment 67. The polymer microcapsule as defined in Embodiment 63 wherein the polymer microcapsule encapsulating an insecticide has a particle size D90 of less than 10 microns.

Embodiment 68. A polymer microcapsule prepared as defined in any one of Embodiments 37 through 67 from the reaction product as defined in any one of Embodiments 1 through 36 wherein the polymer microcapsule encapsulates an agrochemical.

Embodiment 69. The polymer microcapsule of Embodiment 68 wherein the agrochemical is a herbicide or pheromone.

Embodiment 70. The polymer microcapsule of Embodiment 69 wherein the agrochemical is a herbicide.

Embodiment 71. The polymer microcapsule of Embodiment 70 wherein the herbicide is bixlozone, broclozone or clomazone.

Embodiment 72. The polymer microcapsule of Embodiment 68 wherein the agrochemical is a pheromone.

Embodiment 73. The polymer microcapsule of Embodiment 72 wherein the pheromone is (Z)-11-Hexadecenal or (Z)-9-tetradecenyl acetate.

Embodiment 74. The polymer microcapsule of any of Embodiments 37 through 73 wherein the polymer is prepared by a technique selected from the group consisting of solvent evaporation, in-situ polymerization, interfacial polymerization, emulsion polymerization, simple and complex coacervation, layer-by-layer deposition, liposomes, interfacial crosslinking, spray coating, pan coating, and combinations thereof.

Embodiment 75. The polymer microcapsule of Embodiment 74, wherein the polymer is prepared by interfacial polymerization.

Embodiment 76. The polymer microcapsule of Embodiment 75 wherein the polymer is prepared by the process comprising:
a) forming a mixture comprising:
   the reaction product as described in any of Embodiments 1 through 36; and
   at least one di-amine or tri-amine compound;
b) reacting the reaction product as described in any of Embodiments 1 through 36 and the at least one di-amine or tri-amine compound to form the polymer secondary reaction product of the reaction product as described in any of Embodiments 1 through 36 of Formula 1 and the at least one di-amine or tri-amine compound in the presence of an agrochemical compound; and
c) microencapsulating the agrochemical compound with the secondary reaction product.

Embodiment 77. The polymer microcapsule of Embodiment 76, wherein the agrochemical compound is selected from a herbicide, herbicide safener, fungicide, insecticide, nematocide, bactericide, acaricide, growth regulator, pheromone, and plant nutrient.

Embodiment 78. The polymer microcapsule of Embodiment 77, wherein the agrochemical compound is selected from a herbicide, fungicide, insecticide and pheromone.

Embodiment 79. The polymer microcapsule of Embodiment 78, wherein the agrochemical compound is a herbicide.

Embodiment 80. The polymer microcapsule of Embodiment 78, wherein the agrochemical compound is an insecticide.

Embodiment 81. The polymer microcapsule of Embodiment 78, wherein the agrochemical compound is a pheromone.

Embodiment 82. The agrochemical composition described in any of Embodiments 37 through 81, wherein the agrochemical composition is in a form selected from capsule suspension concentrates (CS), mixtures of suspension concentrates (SC) and capsule suspension concentrates (CS) formulations (ZC), flowable concentrates for seed treatment (FS), granules (GR), mixed formulations of capsule suspension concentrates (CS) and aqueous suspo-emulsions (SE) formulations (ZE), mixtures of capsule suspension concentrates (CS) and oil-in-water emulsions (EW) formulations (ZW), wettable powders (WP), and combinations thereof.

Embodiment 83. The agrochemical composition described in Embodiment 82, wherein the agrochemical composition is in the form of a CS formulation or a ZC formulation.

Embodiment 84. The agrochemical composition described in Embodiment 83 wherein the agrochemical composition is in the form of a CS formulation.

Embodiment 85. The agrochemical composition described in Embodiment 83 wherein the agrochemical composition is in the form of a ZC formulation.

Embodiment 86. The agrochemical composition described in Embodiment 82 wherein the agrochemical composition is in the form of a FS formulation.

Embodiment 87. The agrochemical composition described in Embodiment 82 wherein the agrochemical composition is in the form of a GR formulation.

Embodiment 88. The agrochemical composition described in Embodiment 82 wherein the agrochemical composition is in the form of a ZE formulation.

Embodiment 89. The agrochemical composition described in Embodiment 82 wherein the agrochemical composition is in the form of a ZW formulation.

Embodiment 90. The agrochemical composition described in Embodiment 82 wherein the agrochemical composition is in the form of a WP formulation.

Embodiments of this invention, including Embodiments 1-90 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-90 above as well as any other embodiments described herein, and any combination thereof, pertain to the compositions comprising a compound of Formula 1 and methods of using the compound of Formula 1 of the present invention.

Combinations of Embodiments 1-90 are illustrated by:

Embodiment PA. The reaction product of the tri-ol compound of Formula 1 in the Summary of the Invention wherein
m is 1 to 3;
n is 1 to 3;
R¹ is selected from H and linear C₁-C₆ alkyl; and
R² is selected from linear and branched C₂-C₆ alkyl and saturated or unsaturated C₅ cycloalkyl, optionally substituted with a linear C₁-C₆ alkyl.

Embodiment PB. The reaction product of the tri-ol compound of Embodiment PA wherein
m is 1 to 2;
n is 1 to 2;
R¹ is selected from H, —CH₃, —CH₂CH₃ and —CH₂CH₂CH₃; and
R² is selected from linear C₂-C₆ alkyl and saturated C₅ cycloalkyl, optionally substituted with a linear C₁-C₃ alkyl.

Embodiment PC. The reaction product of the tri-ol compound of Embodiment PB wherein
m is 1;
n is 1;
R¹ is selected from H, —CH₃ and —CH₂CH₃; and
R² is selected from linear C₂-C₄ alkyl and saturated C₅ cycloalkyl, optionally substituted with CH₃.

Embodiment PD. The reaction product of the tri-ol compound the Summary of the Invention or any one of Embodiments PA through PC wherein the at least one starter di-isocyanate compound is aliphatic, cycloaliphatic, or aromatic.

Embodiment PE. The reaction product of the tri-ol compound of Embodiment PD wherein the at least one starter di-isocyanate compound is selected from 1,6-hexamethylene diisocyanate (HDI), 2,2,4- and 2,4,4-trimethyl-1,6-hexamethylene diisocyanate (TMDI), 1-isocyanato-3,3,5-trimethyl-5-isocyanatomethylcyclohexane (isophorone diisocyanate or IPDI), perhydro-2,4′- and -4,4′-diphenylmethane diisocyanate (HMDI), 1,4-diisocyanato-2,2,6-trimethylcyclohexane (TMCDI), 1,3- and 1,4-bis(isocyanato methyl)cyclohexane, m-xylylene diisocyanate (m-XDI), p-xylylene diisocyanate (p-XDI), m- and p-tetramethyl-1,3- and -1,4-xylylene diisocyanate (m- and p-TMXDI), 2,4- and 2,6-tolylenediisocyanate (TDI), 4,4′-2,4′- and 2,2′-diphenylmethane diisocyanate (MDI), polymeric MDI (PMDI), homologues thereof, isomers thereof, and mixtures thereof.

Embodiment PF. A polymer microcapsule prepared from the reaction product of the Summary of the Invention or any one of Embodiments PA through PE and an amine selected from a di-amine or a tri-amine.

Embodiment PG. The polymer microcapsule of Embodiment PF wherein wherein the di-amine is selected from the group consisting of ethylenediamine (EDA), 1,2-propanediamine, 1,3-propanediamine (DAP), 2-methyl-1,2-propanediamine, 2,2-dimethyl-1,3-propanediamine, 1,3-butanediamine, 1,4-butanediamine, 1,3-pentanediamine, 1,5-pentanediamine, 2-butyl-2-ethyl-1,5-pentanediamine, 1,6-hexamethylenediamine (HAMDA), methylbis(3-aminopropyl)amine, 1,5-diamino-2-methylpentane (MPMD), 1,2-diaminocyclohexane, 1,3-diaminocyclohexane, 1,4-diaminocyclohexane, bis(4-aminocyclohexyl)methane, bis(4-amino-3-methylcyclohexyl)methane, 1-amino-3-aminomethyl-3,5,5-trimethylcyclohexane (isophoronediamine or IPDA), m-xylylenediamine (XDA), 1,3-bis(aminomethyl)cyclohexane (BAC), methylene-bis(cyclohexylamine) (MBCHA), 4,4′-methylenebis(2-methylcyclohexylamine) (MBMCHA), homologues thereof, isomers thereof, and combinations thereof.

Embodiment PH. The polymer microcapsule of Embodiment PG wherein the di-amine compound comprises 1,6-hexamethylenediamine (HMDA).

This invention also relates to a method for controlling undesired vegetation comprising applying to the locus of the vegetation agriculturally effective amounts of the compounds of the invention (e.g., as a composition described herein). Of note as embodiments relating to methods of use are those involving the compounds of embodiments described above.

Microencapsules formed with pre-polymers of the invention can be used to microencapsulate agriculturally active compounds for the selective control of pests in crops such as wheat, barley, maize, soybean, sunflower, cotton, oilseed rape and rice, and specialty crops such as sugarcane, citrus, fruit and nut crops.

The compound of Formula 1 can be prepared by general methods known in the art of synthetic organic chemistry. The following method (or any variation thereof) as described in Scheme 1 can be used to prepare the compound of Formula 1. Alternatively, the compound of Formula 1 may be commercially available. The definitions of m, n, R¹ and R² in the compound of Formula 1 below are as defined in the Summary of the Invention unless otherwise noted.

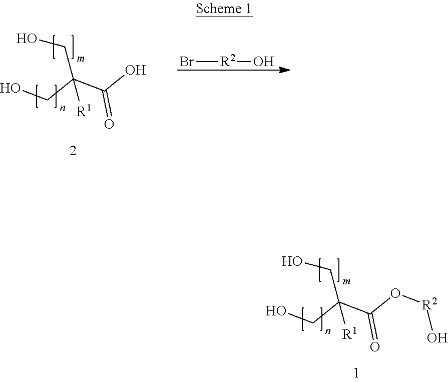

Compounds of Formula 1 can be prepared by general methods known in the art of synthetic organic chemistry as shown above in Scheme 1. Preparation of a tri-ol compound of Formula 1 (Scheme 1) can be accomplished by the esterification reaction of one molar equivalent of an acid compound of Formula 2 with at least one molar equivalent of a bromoalkanol. An example of this esterification reaction of this type can be found in the literature (e.g., Angew. Chem. Int. Ed. 2017, 56, 16664-16668).

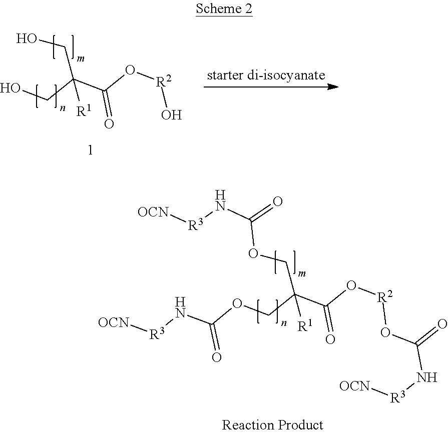

The reaction product can be prepared with one mole of a triol compound of Formula 1 and at least 3 or more molar equivalents of a starter di-isocyanate compound as shown above in Scheme 2. The reaction can be achieved in various organic solvents and at various temperatures ranging from ambient to the reflux temperature of the solvent. The “reaction product” will typically possess three reactive isocyanate functional groups. The “reaction product” is alternatively referred to as the “tri-isocyanate” or “pre-polymer”. An example of this reaction can be found in Step B of Synthesis Example 1. Other conditions can be found, for example, in the literature, such as US 2016/0340563A1.

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products.

The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Polymer microcapsules formed from the pre-polymer compounds of the present invention can be prepared by several methods, including interfacial polymerization. The process involves bringing the pre-polymer into contact with a di-amine or triamine where the pre-polymer is solubilized in an organic (i.e. water-immiscible solvent) layer, and the di-amine or triamine dissolved in an aqueous layer. Polymerization (i.e. microcapsule formation) occurs at the interface between a biphase mixture of said organic (i.e. water-immiscible) and said aqueous layers. The solvent containing the pre-polymer of the present invention comprises at least one water-immiscible solvent (or mixtures thereof), which has a solubility in water of less than 5% by weight at 25 °C. Suitable water-immiscible solvents include C8 to C11 aromatic petroleum derivatives (aromatic hydrocarbons) or commercially available solvents under the following brand names: Solvesso® 100, Solvesso® 150, Solvesso® 200, Solvesso® 150ND, Solvesso® 200ND. Additional suitable water-immiscible solvents include vegetable and seed oils selected from liquid triglycerides (e.g., olive oil, kapok oil, castor oil, papaya oil, camellia oil, palm oil, sesame oil, corn oil, rice bran oil, peanut oil, walnut oil, coconut oil, cotton seed oil, soybean oil, rapeseed oil, linseed oil, tung oil, sunflower oil, safflower oil, or their transesterification products thereof, e.g., alkyl esters, such as rapeseed oil methyl ester or rapeseed oil ethyl ester). Suitable water-immiscible solvents include animal-derived oils (e.g., whale oil, cod-liver oil, or mink oil). Suitable, liquid esters of C1-C12 monoalcohols or polyols, for example methanol or ethanol, butanol, n-octanol, i-octanol, dodecanol, cyclopentanol, cyclohexanol, cyclooctanol, ethylene glycol, propylene glycol or benzyl alcohol, with C6-C10 carboxylic or polycarboxylic acids, such as caproic acid, capric acid, caprylic acid and pelargonic acid; or with aromatic carboxylic acids such as benzoic acid, toluic acid, salicylic acid and phthalic acid; hydrocarbons such as aromatic depleted, linear paraffinic, isoparaffinic, cycloparaffinic having a flash point between 40 °C and 250 °C and a distillation range between 150 °C and 450 °C, esters, such as terpenoid esters (for example isobornyl acetate), benzyl acetate, benzyl benzoate, butyl benzoate; phosphate ester such as tributyl phosphate, trioctyl phosphate; and liquid amides of C₁-C₃ amines, alkylamines or alkanolamines with C6-C18 carboxylic acids; or mixtures thereof such as N,N-dialkyl alkylamides, preferably fatty acid dimethylamides, more preferably N,N-dimethyl octanamide and/or N,N-dimethyl decanamide and/or N,N-dimethyl dodecanamide and n-octyl-2-pyrrolidone (NOP), N-decyl-2-pyrrolidone (NDP), N-dodecyl-2-pyrrolidone (NDDP), and the morpholine C6-12 Acyl amide derivatives (i.e. the morpholine C8-C10 fatty acids such as JEFFSOL® AG-1730 from Huntsman). Preferred solvents are selected from Solvesso® 200ND), benzyl acetate, N,N-dimethyl dodecanamide, and JEFFSOL® AG-1730.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “d” means doublet, “t” means triplet, “m” means multiplet, and “dd” means doublet of doublets. Mass spectra (MS) are reported as the molecular weight of the highest isotopic abundance parent ion (M+1) formed by addition of H+ (molecular weight of 1) to the molecule, or (M-1) formed by the loss of H+ (molecular weight of 1) from the molecule, observed by using liquid chromatography coupled to a mass spectrometer (LCMS) using atmospheric pressure chemical ionization (AP+).

### SYNTHESIS EXAMPLE 1

### Step A: Preparation of 3-Hydroxypropyl 3-Hydroxy-2-(Hydroxymethyl)-2-Methylpropanoate

In a round bottom flask dimethylolpropionic acid (CAS No. 4767-03-7, 10.03 g, 74.76 mmol) and potassium hydroxide (5.45 g, 97.19 mmol) was heated to 100 °C in N,N-dimethylformamide (56 mL) under nitrogen with magnetic stirring. During heating, the heterogeneous mixture became a colorless solution. When the reaction reached 100 °C, an exotherm to 113 °C was observed. The reaction mass was stirred for 2 h at 100 °C. The reaction temperature was then reduced to 45 °C and 3-bromo-1-propanol (12.47 g, 89.72 mmol) was added dropwise. The resulting pale-yellow reaction mass was stirred at 45 °C and after a period of time, the reaction mass turned cloudy due to precipitated potassium bromide and allowed to stir for 16 h at 45 °C. Upon completion of the reaction, the reaction mixture was concentrated on a rotary evaporator to remove N,N-dimethylformamide at 70 °C. The cloudy residue was triturated with isopropanol, stirred for 15 min, and filtered. The filtrate was concentrated on a rotary evaporator at 70 °C to obtain the desired compound (14.7 g, 76.47 mmol) as a thick, colorless oil. The spectroscopic analysis of the oil is in accordance with the title compound, a tri-ol of the present invention.

¹H NMR (500 MHz, DMSO-d₆) δ 4.56 (m, 2H), 4.42 (m, 1H), 4.05 (t, 2H), 3.38 (m, 7H), 1.70 (q, 2H), 1.03 (s, 3H).

### Step B: Preparation of Compound 1

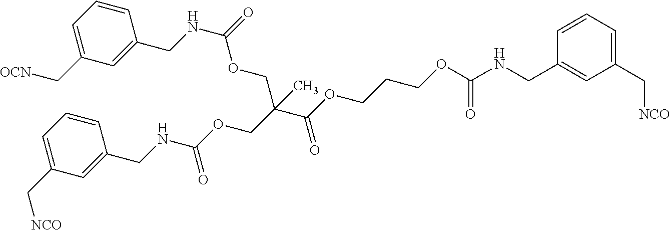

m-Xylylene diisocyanate (m-XDI, 13.9 g, 73.84 mmol) was placed in a 60 mL vial fitted with a condenser and a N₂ inlet, and heated to 55 °C. A solution of 3-hydroxypropyl 3-hydroxy-2-(hydroxymethyl)-2-methylpropanoate (i.e. the product of Step A, 1.58 g, 8.21 mmol) in acetophenone (16 mL) was prepared with stirring. The cloudy solution was added dropwise via pipet over 40 min to the heated m-xylylene diisocyanate (m-XDI). The resulting pale-yellow solution was stirred at 55 °C for 60 min. Upon completion of the reaction, cyclohexane (30 mL) was added to reaction at 55 °C. The reaction became cloudy, and the mixture was stirred for 5 min at 55 °C. Stirring was stopped and the mass allowed to separate the layers. The top layer was discarded, and the viscous bottom layer containing the product was kept. The cyclohexane addition (50 mL) and layer separation was repeated three times. The viscous oil was dissolved in Agnique AMD 10 (18.6 g) to prepare a 25 wt % of the desired product in solvent. MS (M+H)=756.3

The microcapsules of this invention will generally be used in an agricultural composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

The general types of solid compositions are dusts, powders, granules, pellets, prills, pastilles, tablets, filled films (including seed coatings) and the like, which can be water-dispersible (“wettable”) or water-soluble. Active ingredient can be microencapsulated and further formed into a suspension or solid formulation; alternatively the entire formulation of active ingredient can be microencapsulated (or “overcoated”). Microencapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

Compositions in accordance with the present disclosure may be formulated with further suitable formulation components known in the art. For example, compositions in accordance with the present disclosure may be formulated using known auxiliaries, adjuvants, diluents, solvents, dispersants, surfactants, protective colloids, thickeners, penetrating agents, stabilizers, sequestering agents, anti-caking agents, coloring agents, pigments, corrosion inhibitors, biocides, antifoamers, antifreeze agents, rheology modifiers, and combinations thereof.

The term surfactant, as used herein, means an agriculturally acceptable material which imparts emulsifiability, stability, spreading, wetting, dispersibility or other surface-modifying properties. Examples of suitable surfactants include lignin sulfonates, fatty acid sulfonates (e.g. lauryl sulfonate), the condensation product of formaldehyde with naphthalene sulfonate, alkylarylsulfonates, ethoxylated alkylphenols, and ethoxylated fatty alcohols. Other known surfactants that have been used with herbicides are also acceptable.

Suitable diluents may be added during the formulation process, after the formulation process (e.g. by the user—a farmer or custom applicator), or both. The term diluent includes all liquid and solid agriculturally acceptable material-including carriers which may be added to the herbicides to bring them in a suitable application or commercial form and include solvents, emulsifiers, and dispersants. Examples of suitable solid diluents or carriers are aluminum silicate, talc, calcined magnesia, kieselguhr, tricalcium phosphate, powdered cork, absorbent carbon black, chalk, silica, and clays such as kaolin and bentonite. Examples of suitable liquid diluents include water, organic solvents (e.g. acetophenone, cyclohexanone, isophorone, toluene, xylene, petroleum distillates), amines (e.g., ethanolamine, dimethylformamide), and mineral, animal, and vegetable oils (used alone or in combination).

Compositions in accordance with the present disclosure may also be formulated with further suitable additional active ingredients known in the art. An additional active ingredient may be utilized if broadening of the spectrum of control or preventing the build-up of resistance is desired. Suitable additional active ingredients include the any further herbicide, herbicide safener, insecticide, fungicide, nematocide that does not extinguish the biological activity of the compositions. Suitable additional active ingredients can also be formulated outside the compositions (e.g., separate from the microcapsules, but within the same formulation).

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of lnsecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof, amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as NN-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

In one common embodiment, microencapsules of the invention are applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Plant varieties and cultivars of the desired vegetation in the locus treated with a compound of the invention can be obtained by conventional propagation and breeding methods or by genetic engineering methods. Genetically modified plants (transgenic plants) are those in which a heterologous gene (transgene) has been stably integrated into the plant's genome. A transgene that is defined by its particular location in the plant genome is called a transformation or transgenic event.

A pre-polymer of this invention can also be used to microencapsulate other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection.

Examples of herbicide active ingredients are allidochlor, acetochlor, acifluorfen and its sodium salt, aclonifen, acrolein (2-propenal), alachlor, alloxydim, ametryn, amicarbazone, amidosulfuron, aminocyclopyrachlor and its methyl and ethyl esters and its sodium and potassium salts, aminopyralid, amitrole, ammonium sulfamate, anilofos, asulam, atrazine, azimsulfuron, beflubutamid, beflubutamid-M, benazolin, benazolin-ethyl, bencarbazone, benfluralin, benfuresate, benquinotrione, bensulfuron-methyl, bensulide, bentazone, benzobicyclon, benzofenap, bicyclopyrone, bifenox, bilanafos, bipyrazone, bispyribac and its sodium salt, bixlozone, broclozone, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinflubrolin, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyranil, cyclopyrimorate, cyclosulfamuron, cycloxydim, cyhalofop-butyl, cypyrafluone, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimepyrolimet, dimesulfazet, dimethachlor, dimethametryn, dimethenamid, dimethenamid-P, dimethipin, dimethylarsinic acid and its sodium salt, dinitramine, dinoterb, dioxopyritrione, diphenamid, diquat dibromide, dithiopyr, diuron, DNOC, endothal, EPTC, epyrifenacil, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenpyrazone, fenquinotrione, fentrazamide, fenuron, fenuron-TCA, feproxydim, flamprop-methyl, flamprop-M-isopropyl, flamprop-M-methyl, flazasulfuron, florasulam, florpyrauxifen, florpyrauxifen-benzyl, florpyrauxifen-methyl, fluazifop-butyl, fluazifop-P-butyl, fluazolate, flucarbazone, flucetosulfuron, fluchloralin, fluchloraminopyr, fluchloraminopyr-tefuryl, flufenazopyr, flufenacet, flufenoximacil, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, flusulfinam, fluthiacet-methyl, fomesafen, foramsulfuron, fosamine-ammonium, glufosinate, glufosinate-ammonium, L-glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, icafolin, icafolin-methyl, imazamethabenz-methyl, imazamox, imazapic, imazapyr, imazaquin, imazaquin-ammonium, imazethapyr, imazethapyr-ammonium, imazosulfuron, indanofan, indaziflam, indolauxipyr, indolauxipyr-cyanomethyl, iofensulfuron, iofensulfuron-sodium, iodosulfuron-methyl, iodosulfuron-sodium, ioxynil, ioxynil octanoate, ioxynil-sodium, ipfencarbazone, iptriazopyrid, isoproturon, isouron, isoxaben, isoxaflutole, isoxachlortole, lactofen, lancotrione, lancotrione-sodium, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metproxybicyclone, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prochlorosulfone, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyraquinate, pyrasulfotole, pyrazogyl, pyrazolynate, pyrazoxyfen, pyrazosulfuron-ethyl, pyribenzoxim, pyributicarb, pyridate, pyriflubenzoxim, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimisoxafen, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, tetflupyrolimet, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tripyrasulfone, tritosulfuron, vernolate, 3-(2-chloro-3,6-difluorophenyl)-4-hydroxy-1-methyl-1,5-naphthyridin-2(1H)-one, 6-chloro-4-(2,7-dimethyl-1-naphthalenyl)-5-hydroxy-2-methyl-3(2H)-pyridazinone, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, 2-methyl-3-(methylsulfonyl)-N-(1-methyl-1H-tetrazol-5-yl)-4-(trifluoromethyl)benzamide, 2-methyl-N-(4-methyl-1,2,5-oxadiazol-3-yl)-3-(methylsulfinyl)-4-(trifluoromethyl)benzamide, methyl 2-[2-[2-bromo-5-[3,6-dihydro-3-methyl-2,6-dioxo-4-(trifluoromethyl)-1(2H)-pyrimidinyl]-4-fluorophenoxy]phenoxy]-2-methoxyacetate, isoxafenacil (ethyl 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-isoxazolecarboxylate), 1-(2-carboxyethyl)-4-(2-pyrimidinyl)pyridazinium and its salts and esters, 5-oxo-1-[(2,3-difluorophenyl)methyl]-proline, 5-oxo-1-[(2,3,4-trifluorophenyl)methyl]-proline, L-valine, N-[2-[(2R)-1-[(2,3-difluorophenyl)methyl]-5-oxo-2-pyrrolidinyl]acetyl]-, methyl ester, L-valine, N-[2-[(2S)-1-[(2,3-difluorophenyl)methyl]-5-oxo-2-pyrrolidinyl]acetyl]-, methyl ester, cyclopropanepropanoic acid, α-[[2-[(2R)-1-[(2,3-difluorophenyl)methyl]-5-oxo-2-pyrrolidinyl]acetyl]amino]-, methyl ester, (aS)-, cyclopropanepropanoic acid, and α-[[2-[(2S)-1-[(2,3-difluorophenyl)methyl]-5-oxo-2-pyrrolidinyl]acetyl]amino]-, methyl ester, (aS)-.

Of note is a herbicide selected from the group consisting of bixlozone, broclozone, carfentrazone, carfentrazone-ethyl, chlorimuron, chlorimuron-ethyl, clomazone, florasulam, flupyrsulfuron, flupyrsulfuron-methyl, fluthiacet, fluthiacet-methyl, mesotrione, metolachlor, S-metolachlor, pinoxaden, pyroxasulfone, pyroxsulam, rimisoxafen, sulfentrazone, tembotrione, tetflupyrolimet, thifensulfuron, thifensulfuron-methyl, tribenuron and tribenuron-methyl.

The pre-polymer compounds of the present invention can also be used to microencapsulate herbicide safeners selected from the group consisting of benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

The pre-polymer compounds of the present invention can also be used to miciroencapsulate pheromones. Examples of pheromones include Formaldehyde; 2,2-Dibromoacetaldehyde; Acetaldehyde; 2-Methyl-2-propenal; 2-Methylpropanal; 2-Propenal; 3,3-Dibromo-2-propenal; Propanal; 2-Butenal; 2-Methyl-2-butenal; 2-Methylbutanal; 2-Methylenebutanal; 3-Methyl-2-butenal; 3-Methyl-3-butenal; 3-Methylbutanal; Butanal; (E)-2-Pentenal; 2-Methylenepentanal; 2-Pentenal; 3-Methyl-1-(vinyloxy)-butane; 4-Methylpentanal; 4-Pentenal; 5-Methylfurfural; Furan-2-carbaldehyde; Pentanal; (E)-2-Hexenal; (E)-2-Methyl-2-hexenal; (E)-3-Hexenal; (E)-4-oxo-2-Hexenal; (E,E)-2,4-Dimethyl-2,4-hexadienal; (E,E)-2,4-Hexadienal; (Z)-2-Hexenal; (Z)-3-Hexenal; (Z)-4-oxo-2-Hexenal; 1-Hexenal; 2,3-Dihydroxybenzaldehyde; 2-Hexenal; 3-((E)-2-Hexenoxy)-hexanal; 3,5-Dimethylhexanal; 3-Ethoxyhexanal; 3-Hydroxybenzaldehyde; 3-Hydroxyhexanal; 4-Hydroxy-3,5-dimethoxybenzaldehyde; 4-Hydroxybenzaldehyde; 5-Methylhexanal; Hexanal; (1R,2S,5R)-2-Methyl-5-((R)-1-oxopropan-2-yl)-cyclopentanecarbaldehyde; (1R,2S,5S)-2-Methyl-5-((R)-1-oxopropan-2-yl)-cyclopentanecarbaldehyde; (1R,5S)-6,6-Dimethylbicyclo[3.1.1]hept-2-ene-2-carbaldehyde; (1S,2S,5R)-2-Methyl-5-((R)-1-oxopropan-2-yl)-cyclopentanecarbaldehyde; (3S,8R)-2-Methyl-5-(1-formylethyl)-1-cyclopentene-1-carbaldehyde; (3S,8S)-2-Methyl-5-(1-formylethyl)-1-cyclopentene-1-carbaldehyde; (5S,8S)-2-Methyl-5-(1-formylethyl)-1-cyclopentene-1-carbaldehyde; (E)-2-(2-Hydroxyethyl)-6-methyl-2,5-heptadienal; (E)-2-(2-Hydroxyethylidene)-6-methyl-5-heptenal; (E)-2-Heptenal; (E)-2-Isopropyl-5-methyl-2-hexenal; (E)-2-Methyl-2-heptenal; (E,Z)-2,4-Heptadienal; (R)-2,6-Dimethyl-5-heptenal; (S)-4-(Prop-1-en-2-yl)-cyclohex-1-enecarbaldehyde; (Z)-2-Isopropyl-5-methyl-2-hexenal; (Z,Z)-2,4-Heptadienal; 2-(3-Methylcyclopentyl)-propanal; 2-(3-Methylcyclopentyl)-propanal; 2,3,6-Tribromo-4,5-dihydroxybenzaldehyde; 2,3-Dibromo-4,5-dihydroxybenzaldehyde; 2,6-Dimethyl-5-heptenal; 2-Methoxybenzaldehyde; 2-Methyl-1-cyclopentenecarboxaldehyde; 2-Methyl-2-heptenal; 2-Methyl-5-(1-oxopropan-2-yl)-cyclopentanecarbaldehyde; 2-Methylcyclopent-1-enecarbaldehyde; 3,3-Dimethyl-5-oxo-7-oxabicyclo[4.1.0]heptane-1-carbaldehyde; 3,4-Dimethylbenzaldehyde; 3,5-Dibromo-4,5-dihydroxybenzaldehyde; 3,5-Dibromo-4-hydroxybenzaldehyde; 3-Bromo-4,5-dihydroxybenzaldehyde; 3-Bromo-4-hydroxybenzaldehyde; 3-Bromo-5-hydroxy-4-methoxybenzaldehyde; 3-Hydroxybenzene-1,2-dicarbaldehyde; 3-Methylbenzaldehyde; 4-(Heptyloxy)-butanal; 4-Methoxybenzaldehyde; 5-(1-Formylethyl)-2-methyl-2-cyclopentene-1-carbaldehyde; 6-Methyl-5-heptenal; 6-Methylheptanal; Benzaldehyde; Cartilagineal; Cyclohexanedial; Heptanal; Taxifolial D; (1R,2S)-cis-2-Isopropenyl-1-methylcyclobutaneethanal; (1S,2R,3S)-2-(1-Formylvinyl)-5-methylcyclopentanecarbaldehyde; (1S,2S,3S)-2-(1-Formylvinyl)-5-methylcyclopentanecarbaldehyde; (2Z,6E)-8-Chloro-6-chloromethyl-2-methyl-2,6-octadienal; (4S)-(3-Oxoprop-1-en-2-yl)-cyclohex-1-enecarbaldehyde; (E)-(3,3-Dimethyl)-cyclohexylideneacetaldehyde; (E)-2-(3,3-Dimethylcyclohexylidene)-acetaldehyde; (E)-2-(4-Methyl-3-pentenyl)-butenedial; (E)-2-(4-Methyl-3-pentenylidene)-butanedial; (E)-2,7-Octadienal; (E)-2-Methyl-2-octenal; (E)-2-Methyl-5-(3-furyl)-2-pentenal; (E)-2-Octenal; (E)-3,7-Dimethyl-2,6-octadienal; (E)-3,7-Dimethyl-2,6-octadienal; (E)-3-Octenal; (E)-4-oxo-2-Octenal; (E)-7-Methyl-2-octenal; (E,E)-2,4-Octadienal; (E,E)-2,6-Dimethyl-8-hydroxy-2,6-octadienal; (E,E)-2,6-Octadienal; (E,E)-2,6-Octadienedial; (E,Z)-2,4-Octadienal; (E,Z)-2,6-Octadienal; (R)-1,2-Dimethyl-3-methylenecyclopentyl-acetaldehyde; (R)-3,7-Dimethyl-6-octenal; (Z)-(3,3-Dimethyl)-cyclohexylideneacetaldehyde; (Z)-2-(3,3-Dimethylcyclohexylidene)-acetaldehyde; (Z)-3,7-Dimethyl-2,6-octadienal; (Z,E)-3,7-Dimethyl-2,6-octadienal; 1-Octenal; 2-(1-Formylvinyl)-5-methylcyclopentanecarbaldehyde; 2-(3,4-Dihydroxyphenyl)-2-oxoacetaldehyde; 2,6,6-Trimethyl-1-cyclohexene-1-carbaldehyde; 2-Ethyloctanal; 2-Hydroxy-6-methylbenzaldehyde; 2-Methyl benzaldehyde; 2-Methyl-5-(1-formylethyl)-1-cyclopentene-1-carbaldehyde; 2-Octenal; 2-Phenylacetaldehyde; 2-Phenylpropenal; 3,4-Dihydroxyphenylglyoxal; 3,7-Dimethyl-6-octenal; 3-Ethoxy-4-hydroxybenzaldehyde; 3-Ethyl benzaldehyde; 3-Isopropyl-6-methyl benzaldehyde; 3-Octenal; 3-oxo-4-Isopropylidene-1-cyclohexene-1-carboxyaldehyde; 4-Ethylbenzaldehyde; 4-Hydroxy-2-methyl benzaldehyde; 4-Hydroxy-3-methoxybenzaldehyde; 4-Isopropenyl-1-cyclohexene-1-carbaldehyde; 4-Isopropenyl-3-oxo-1-cyclohexene-1-carboxyaldehyde; 4S-4-Isopropenyl-3-oxo-1-cyclohexene-1-carboxyaldehyde; 5-Ethylcyclopent-1-ene-carbaldehyde; 6,6-Dimethylbicyclo[3.1.1]hept-2-ene-2-carbaldehyde; 6-Methyloctanal; 7-Methyloctanal; Anisomorphal; cis-2-Isopropenyl-1-methylcyclobutaneethanal; Octanal; Peruphasmal; (1R,2S,6R)-2,6-Dimethyl-3-oxabicyclo[4.2.0]octane-2-carbaldehyde; (E)-2-Methyl-2-nonenal; (E)-2-Nonenal; (E)-3-Phenyl-2-propenal; (E)-4,8-Nonadienal; (E)-8-Methyl-2-nonenal; (E,E)-2,4-Nonadienal; (E,E,E)-2,4,6-Nonatrienal; (E,E,Z)-2,4,6-Nonatrienal; (E,Z)-2,6-Nonadienal; (E,Z,Z)-2,4,6-Nonatrienal; (Z)-2-Methyl-2-nonenal; (Z)-3-Nonenal; (Z)-4,8-Nonadienal; (Z)-4-Nonenal; (Z)-8-Methyl-2-nonenal; 2,6-Nonadienal; 2-Formyl-3-methylcyclopenteneacetaldehyde; 2-Nonenal; 2-Phenyl-2-butenal; 3-(4-Methoxyphenyl)-2-propenal; 3,5-di-tert-Butyl-4-hydroxybenzaldehyde; 3-Phenyl-2-propenal; 3-Phenylpropanal; 6-Ethyl benzaldehyde; 7,7-Dimethylbicyclo[4.1.0]hept-3-ene-3-carbaldehyde; 7-Methylnonanal; 8-Methylnonanal; 9-Acetyloxynonanal; Gibepyrone C; Nonanal; (4R,8R)-4,8-Dimethyldecanal; (4R,8S)-4,8-Dimethyldecanal; (E)-17,18,19,20-Tetranorloba-8,10,13(15)-trien-16-al; (E)-2,9-Decadienal; (E)-2-Decenal; (E)-2-Methyl-2-decenal; (E)-2-Methyl-3-(2,3-dibromo-4,5-dihydroxyphenyl)-propenal; (E)-4-oxo-2-Decenal; (E)-8-Hydroxy-4,8-dimethyl-4,9-decadienal; (E)-9-Methyl-2-decenal; (E,E)-2,4-Decadienal; (E,Z)-2,4-Decadienal; (Z)-4-Decenal; (Z)-5-Decenal; (Z)-9-Methyl-2-decenal; (Z,Z)-2,4-Decadienal; 1-Decenal; 2-Decenal; 2-Ethyldecanal; 3-(2,3-Dibromo-4,5-dihydroxyphenyl)-2-methylpropanal; 4,5-Dimethyldecanal; 4,8-Dimethyldecanal; Caraibical; Decanal; Rogiolal; (2E,4E)-2,6,10-Trimethylundeca-2,4,9-trienal; (2E,4E,7Z)-2,6,10-Trimethylundeca-2,4,7,9-tetraenal; (5E)-2,6,10-Trimethylundeca-5,9-dienal; (E)-2-Undecenal; (E)-6-Ethyl-2,10-dimethyl-5,9-undecadienal; (Z)-4-Undecenal; 10-Undecenal; 2-Butyl-2-octenal; 2-Undecenal; 3-Isopropyl-6-methyl-10-oxoundeca-2,6-dienal; 5-Methyl-2-phenyl-2-hexenal; 8-Isopropyl-5-methyl-3,4,4a,5,6,7,8,8a-octahydronaphthalene-2-carbaldehyde; Austrodoral; Oxytoxin 1; syn-4,6-Dimethylundecanal; Taxifolial A; Taxifolial B; Taxifolial C; Undecanal; (1R,6R,7S,1OR)-1-Hydroxy-4-cadinen-15-al; (2R,7S,11R)-7-Acetoxy-2-hydroxynardosin-1(10)-en-12-al; (3R,5R,9R)-3,5,9-Trimethyldodecanal; (3S,6E)-7-Ethyl-3,11-dimethyldodeca-6,10-dienal; (9R)-3,5,9-Trimethyldodecanal; (E)-10-Dodecenal; (E)-2-Dodecenal; (E)-3,7,11-Trimethyl-6,10-dodecadienal; (E)-5-Dodecenal; (E)-6-Dodecenal; (E)-7-Dodecenal; (E)-8-Dodecenal; (E)-9,11-Dodecadienal; (E)-9-Dodecenal; (E,E)-3,7,11-Trimethyl-2,6,10-dodecatrienal; (E,E)-7-Ethyl-3,11-dimethyl-2,6,10-dodecatrienal; (E,E)-8,10-Dodecadienal; (E,E,E)-3,7-Dimethyl-8,11-dioxo-2,6,9-dodecatrienal; (E,E,Z)-3,7-Dimethyl-8,11-dioxo-2,6,9-dodecatrienal; (E,Z)-2,6-Dodecadienal; (E,Z)-5,7-Dodecadienal; (E,Z)-7,9-Dodecadienal; (E,Z)-8,10-Dodecadienal; (R)-10-Oxo-isodauc-3-en-15-al; (S,E)-3,7,11-Trimethyl-6,10-dodecadienal; (Z)-2-Methyl-5-((1R,5R,6S)-2,6-dimethylbicyclo[3.1.1]hept-2-en-6-yl)-pent-2-enal; (Z)-5-Dodecenal; (Z)-7-Dodecenal; (Z)-9,11-Dodecadienal; (Z)-9-Dodecenal; (Z,E)-3,7,11-Trimethyl-2,6,10-dodecatrienal; (Z,E)-5,7-Dodecadienal; (Z,E)-7-Ethyl-3,11-dimethyl-2,6,10-dodecatrienal; (Z,E)-8,10-Dodecadienal; (Z,Z)-5,7-Dodecadienal; 10-Methyldodecanal; 2,10-Dibromo-3-chloro-7-chamigrene; 2-Dodecenal; 2-Ethyldodecanal; 2-Formylguaiazulene; 3,7,11-Trimethyl-(E)-6,10-dodecadienal; 5-Hydroxy-8-methoxycalamanen-15-al; 5-Hydroxy-8-methoxycalamenene-15-al; Aplysinal; Debromoaplysinal; Dodecanal; Parahigginol D; Polygodial; Sclerosporal; Sinuketal; syn-4,6-Dimethyldodecanal; trans-Calamenen-13-al; (3R,5S,9R,7E,11E)-3,5,9,11-Tetramethyl-7,11-tridecadienal; (3S,4R,6E,10Z)-3,4,7,11-Tetramethyl-6,10-tridecadienal; (E,E)-3,5,9,11-Tetramethyltrideca-7,11-dienal; (Z)-4-Tridecenal; 13,14,15,16-Tetranorclerod-3-en-12-al; 13-Acetyloxytridecanal; 4,6-bis(4-Methylpent-3-en-1-yl)-6-methylcyclo-1,3-hexadienecarbaldehyde; Acanthodoral; Ancistrodial; Cespitulin F; Isoacanthodoral; Tridecanal; (E)-11,13-Tetradecadienal; (E)-11-Tetradecenal; (E,E)-8,10-Tetradecadienal; (E,Z)-4,9-Tetradecadienal; (E,Z)-8,10-Tetradecadienal; (Z)-11,13-Tetradecadienal; (Z)-11-Tetradecenal; (Z)-5-Tetradecenal; (Z)-7-Tetradecenal; (Z)-8-Tetradecenal; (Z)-9,13-Tetradecadien-11-ynal; (Z)-9-Tetradecenal; (Z,E)-9,11,13-Tetradecatrienal; (Z,E)-9,11-Tetradecadienal; (Z,E)-9,12-Tetradecadienal; (Z,Z)-5,8-Tetradecadienal; (Z,Z)-8,10-Tetradecadienal; (Z,Z)-9,11-Tetradecadienal; 10,12-Tetradecadienal; 2,4-Tetradecadienal; 2-Ethyltetradecanal; 3-oxo-13-Tetradecenal; 3-oxo-Tetradecanal; 5,8-Tetradecadienal; 5-Tetradecenal; Norasperenal A; Norasperenal B; Norasperenal C; Norasperenal D; Sargasal I; Sargasal II; Tetradecanal; (6R)-6-Acetoxidichotoma-3,14-diene-1,17-dial; (6R)-6-Hydroxydichotoma-3,14-diene-1,17-dial; (E,E)-3,7,11,15-Tetramethyl-6,10,14-hexadecatrienal; (E,Z)-6,8-Pentadecadienal; (E,Z)-9,11-Pentadecadienal; (E,Z,Z)-2,6,9-Pentadecatrienal; (Z)-10-Pentadecenal; (Z)-2-Chloropentadec-2-enal; (Z)-6,14-Pentadecadienal; (Z,Z)-6,9-Pentadecadienal; (Z,Z)-9,11-Pentadecadienal; 2-Hexyl-2-decenal; Azamial A; Azamial B; Isopachydictyolal; Pentadecanal; Sinularial A; Umbellacin A; Xeniafaraunol A; (1R)-Pimaral; (E)-10-Hexadecenal; (E)-11-Hexadecenal; (E)-14-Hexadecenal; (E)-14-Methyl-8-hexadecenal; (E)-3,7,11,15-Tetramethyl-2-hexadecenal; (E,E)-10,12-Hexadecadienal; (E,E)-10,14-Hexadecadienal; (E,E)-11,13-Hexadecadienal; (E,E)-9,11-Hexadecadienal; (E,E,E)-10,12,14-Hexadecatrienal; (E,E,E)-3,7,11,15-tetramethyl-2,6,10,14-hexadecatetraenal; (E,E,Z)-10,12,14-Hexadecatrienal; (E,E,Z)-4,6,11-Hexadecatrienal; (E,E,Z,Z)-4,6,11,13-Hexadecatetraenal; (E,Z)-10,12-Hexadecadienal; (E,Z)-11,13-Hexadecadienal; (E,Z)-4,6-Hexadecadienal; (E,Z)-6,11-Hexadecadienal; (E,Z)-8,11-Hexadecadienal; (E,Z)-9,11-Hexadecadienal; (R)-(E)-14-Methyl-8-hexadecenal; (R)-(Z)-14-Methyl-8-hexadecenal; (S)-(E)-14-Methyl-8-hexadecenal; (S)-(Z)-14-Methyl-8-hexadecenal; (Z)-10-Hexadecenal; (Z)-11-Hexadecenal; (Z)-12-Hexadecenal; (Z)-13-Hexadecen-11-ynal; (Z)-14-Methyl-8-hexadecenal; (Z)-3,7,11,15-Tetramethyl-2-hexadecenal; (Z)-3-oxo-9-Hexadecenal; (Z)-7-Hexadecenal; (Z)-9-Hexadecenal; (Z,E)-10,12-Hexadecadienal; (Z,E)-11,13-Hexadecadienal; (Z,E)-7,11-Hexadecadienal; (Z,E)-9,11-Hexadecadienal; (Z,Z)-10,12-Hexadecadienal; (Z,Z)-11,13-Hexadecadienal; (Z,Z)-7,10-Hexadecadienal; (Z,Z)-7,11-Hexadecadienal; (Z,Z)-9,11-Hexadecadienal; (Z,Z,E)-7,11,13-Hexadecatrienal; 11-Hexadecenal; 11-Hexadecynal; 13(16),14-Spongiadien-19-al; 2-Methylhexadecanal; 7-Hexadecenal; 9-Hexadecenal; Deacetyl-dihydro-nor-thuridillonal; Dictyodial A; Dihydro-nor-thuridillonal; Hexadecanal; Keikipukalide A; Keikipukalide B; Keikipukalide C; Keikipukalide D; Keikipukalide E; Nor-thuridillonal; Pseudoplexaural; Pukalide aldehyde; Sanadaol; (E)-2-Tridecyl-2-heptadecenal; (Z)-9-Heptadecenal; 1-Heptadecenal; 2-Heptadecenal; Globostelletin C; Globostelletin D; Heptadecanal; (E)-11-Octadecenal; (E)-13-Octadecenal; (E)-14-Octadecenal; (E)-2-Octadecenal; (E)-6-Octadecenal; (E,E)-11,14-Octadecadienal; (E,Z)-2,13-Octadecadienal; (E,Z)-3,13-Octadecadienal; (E,Z)-6,11-Octadecadienal; (Z)-11-Octadecenal; (Z)-13-Octadecenal; (Z)-9-Octadecenal; (Z,E)-13,15-Octadecadienal; (Z,Z)-11,13-Octadecadienal; (Z,Z)-13,15-Octadecadienal; (Z,Z)-3,13-Octadecadienal; (Z,Z)-9,12-Octadecadienal; (Z,Z,Z)-9,12,15-Octadecatrienal; 11-Octadecenal; 13,15-Octadecadienal; 13-Octadecenal; 16-Methyloctadecanal; 1-Octadecenal; 3,6-Dihydroxy-24-nor-9-oxo-9,11-secocholesta-7,22-dien-11-al; 9-Octadecenal; Methyloctadecanal; Octadecanal; Panicein B2; Panicein B3; Panicein C; (Z)-10-Nonadecenal; (Z)-9-Nonadecenal; 9(11)-Pargueren-16-al; Hyrtiosal; Nonadecanal; (2E,6Z,9Z)-2-Methyl-2,6,9-eicosatrienal; (Z)-11-Eicosenal; 11-Eicosenal; 12,18-di-Episcalaradial; 12b-(3′b-Hydroxybutanoyloxy)-20,24-dimethyl-24-oxo-scalara-16-en-25-al; 12b-(3′b-Hydroxypentanoyloxy)-20,24-dimethyl-24-oxo-scalara-16-en-25-al; 12-Deacetoxy-12-oxo-scalaradial; 12-Episcalaradial; 15-Eicosenal; 1-Eicosenal; 3-Deacetyl-22,23-dihydro-24,28-dehydroluffasterol B; 3-Deacetylluffasterol B; 9-Eicosenal; Anthogorgiene B; Deacetylscalaradial; Eicosadienal; Eicosanal; Mooloolabene A; Mooloolabene B; Scalaradial; and combinations thereof.

Of note as an example of pheromones include (Z)-5-decenyl acetate, dodecanyl acetate, (Z)-7-dodecenyl acetate, (E)-7-dodecenyl acetate, (Z)-8-dodecenyl acetate, (E)-8-dodecenyl acetate, (Z)-9-dodecenyl acetate, (E)-9-dodecenyl acetate, (E)-10-dodecenyl acetate, 11-dodecenyl acetate, (Z)-9,11-dodecadienyl acetate, (E)-9,11-dodecadienyl acetate, (Z)-11-tridecenyl acetate, (E)-11-tridecenyl acetate, tetradecanyl acetate, (E)-7-tetradecenyl acetate, (Z)-8-tetradecenyl acetate, (E)-8-tetradecenyl acetate, (Z)-9-tetradecenyl acetate, (E)-9-tetradecenyl acetate, (Z)-10-tetradecenyl acetate, (E)-10-tetradecenyl acetate, (Z)-11-tetradecenyl acetate, (E)-11-tetradecenyl acetate, (Z)-12-pentadecenyl acetate, (E)-12-pentadecenyl acetate, hexadecanyl acetate, (Z)-7-hexadecenyl acetate, (Z)-11-hexadecenyl acetate, (E)-11-hexadecenyl acetate, octadecanyl acetate, (E,Z)-7,9-dodecadienyl acetate, (Z,E)-7,9-dodecadienyl acetate, (E,E)-7,9-dodecadienyl acetate, (Z,Z)-7,9-dodecadienyl acetate, (E,E)-8,10-dodecadienyl acetate, (E,Z)-9,12-dodecadienyl acetate, (E,Z)-4,7-tridecadienyl acetate, (E,E)-9,11-tetradecadienyl acetate, (Z,Z)-9,12-tetradecadienyl acetate, (Z,Z)-7,11-hexadecadienyl acetate, (E,Z)-7,11-hexadecadienyl acetate, (Z,E)-7,11-hexadecadienyl acetate, (E,E)-7,11-hexadecadienyl acetate, (Z,E)-3,13-octadecadienyl acetate, (E,Z)-3,13-octadecadienyl acetate, (E,E)-3,13-octadecadienyl acetate, decanol, (Z)-6-nonenol, (E)-6-nonenol, dodecanol, (Z)-5-decenol, 11-dodecenol, (Z)-7-dodecenol, (E)-7-dodecenol, (Z)-8-dodecenol, (E)-8-dodecenol, (E)-9-dodecenol, (Z)-9-dodecenol, (E)-9,11-dodecadienol, (Z)-9,11-dodecadienol, (Z,E)-5,7-dodecadienol, (E,E)-5,7-dodecadienol, (E,E)-8,10-dodecadien-1-ol, (E,Z)-8,10-dodecadienol, (Z,Z)-8,10-dodecadienol, (Z,E)-8,10-dodecadienol, (E,Z)-7,9-dodecadienol, (Z,Z)-7,9-dodecadienol, (E)-5-tetradecenol, (Z)-8-tetradecenol, (Z)-9-tetradecenol, (E)-9-tetradecenol, (Z)-10-tetradecenol, (Z)-11-tetradecenol, (E)-11-tetradecenol, (Z)-11-hexadecenol, (Z,E)-9,11-tetradecadienol, (Z,E)-9,12-tetradecadienol, (Z,Z)-9,12-tetradecadienol, (Z, Z)-10,12-tetradecadienol, (Z,Z)-7,11-hexadecadienol, (Z,E)-7,11-hexadecadienol, (E)-14-methyl-8-hexadecen-1-ol, (Z)-14-methyl-8-hexadecen-1-ol, (E,E)-10,12-hexadecadienol, (E,Z)-10,12-hexadecadienol, dodecanal, (Z)-9-dodecanal, tetradecanal, (Z)-7-tetradecenal, (Z)-9-tetradecenal, (Z)-11-tetradecenal, (E)-11-tetradecenal, (E)-11,13-tetradecadienal, (E,E)-8,10-tetradecadienal, (Z,E)-9,11-tetradecadienal, (Z,E)-9,12-tetradecadienal, hexadecanal, (Z)-8-hexadecenal, (Z)-9-hexadecenal, (Z)-10-hexadecenal, (E)-10-hexadecenal, (Z)-11-hexadecenal, (E)-hexadecenal, (Z)-12-hexadecenal, (Z)-13-hexadecenal, (Z)-14-methyl-8-hexadecenal, (E)-14-methyl-8-hexadecenal, (Z,Z)-7,11-hexadecadienal, (Z,E)-7,11-hexadecadienal, (Z,E)-9,11-hexadecadienal, (E,E)-10,12-hexadecadienal, (E,Z)-10,12-hexadecadienal, (Z,E)-10,12-hexadecadienal, (Z,Z)-10,12-hexadecadienal, (Z,Z)-11,13-hexadecadienal, octadecenal, (Z)-11-octadecenal, (E)-13-octadecenal, (Z)-13-octadecenal, (Z)-5-decenyl 3-methylbutanoate, (+) cis-7,8-epoxy-2-methyloctadecane.

Also of note as examples of pheromones include citral; geranial; neral; tetradecan-1-al; pentadecan-1-al; pentadecen-1-al; hexadecan-1-al; (Z)-9-hexadecen-1-al; (Z)-11-hexadecen-1-al; (7E,9E)-undeca-7,9-dien-1-al; (11Z, 13Z)-hexadecadien-1-al; (9Z,12E)-tetradecadien-1-al; (8E,10E)-dodecadien-1-al; (11Z)-hexadecadien-1-al; (9Z)-tetradecen-1-al; 6,10-dimethyl-5,9-undecadien-2-ol; (6E)-7,11-dimethyl-3-methylene-1,6,10-dodecatriene; [1S-(1a,2b,5 a)]-4,6,6-trimethyl-Bicyclo[3.1.1]hept-3-en-2-ol; 10-Hexadecenal; (Z)-10-hexadecenal; (E)-10-hexadecenal; and combinations thereof.

The pre-polymer compounds of the present invention can also be used to microencapsulate insecticides. Examples of insecticides include abamectin, acephate, acequinocyl, acetamiprid, acrinathrin, acynonapyr, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, benzpyrimoxan, bifenthrin, kappa-bifenthrin, bifenazate, bistrifluron, borate, broflanilide, buprofezin, cadusafos, carbaryl, carbofuran, cartap, carzol, chlorfenapyr, chlorfluazuron, chloroprallethrin, chlorpyrifos, chlorpyrifos-e, chlorpyrifos-methyl, chromafenozide, clofentezin, chloroprallethrin, clothianidin, cycloprothrin, cycloxaprid ((5S,8R)-1-[(6-chloro-3-pyridinyl)methyl]-2,3,5,6,7,8-hexahydro-9-nitro-5,8-Epoxy-1H-imidazo[1,2-a]azepine), cyenopyrafen, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dicloromesotiaz, dieldrin, diflubenzuron, dimefluthrin, dimehypo, dimethoate, dimpropyridaz, dinotefuran, diofenolan, emamectin, emamectin benzoate, endosulfan, esfenvalerate, ethiprole, etofenprox, epsilon-metofluthrin, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, fluazaindolizine, flucythrinate, flufenerim, flufenoxuron, flufenoxystrobin (methyl (aE)-2-[[2-chloro-4-(trifluoromethyl)phenoxy]methyl]-α-(methoxymethylene)benzeneacetate), fluensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), flupyrimin, fluvalinate, tau-fluvalinate, fluxametamide, fonophos, formetanate, fosthiazate, gamma-cyhalothrin, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, isocycloseram, kappa-tefluthrin, lambda-cyhalothrin, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiocarb, methomyl, methoprene, methoxychlor, metofluthrin, methoxyfenozide, epsilon-metofluthrin, epsilon-momfluorothrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicotine, nitenpyram, nithiazine, novaluron, noviflumuron, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide, N-[1-(difluoromethyl)cyclopropyl]-2-(3-pyridinyl)-2H-indazole-4-carboxamide, oxamyl, oxazosulfyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), pymetrozine, pyrafluprole, pyrethrin, pyridaben, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, silafluofen, spinetoram, spinosad, spirodiclofen, spiromesifen, spiropidion, spirotetramat, sulprofos, sulfiflumin, sulfoxaflor (N-[methyloxido[1-[6-(trifluoromethyl)-3-pyridinyl]ethyl]-λ⁴-sulfanylidene]cyanamide), tebufenozide, tebufenpyrad, teflubenzuron, tefluthrin, kappa-tefluthrin, terbufos, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), thiacloprid, thiamethoxam, thiodicarb, thiosultap-sodium, tiapyrachlor, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, tyclopyrazoflor, zeta-cypermethrin, Bacillus thuringiensis delta-endotoxins, entomopathogenic bacteria, entomopathogenic viruses or entomopathogenic fungi, can combinations thereof.

Examples of insecticides also include diamides, such as chlorantraniliprole, cyantraniliprole, tetrachlorantraniliprole, bromoantraniliprole, dichlorantraniliprole, tetraniliprole, cyclaniliprole, cyhalodiamide, and flubendiamide The pre-polymer compounds of the present invention can also be used to microencapsulate fungicides. Examples of fungicides include fungicides such as acibenzolar-S-methyl, aldimorph, ametoctradin, aminopyrifen, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bifemetstrobin, bitertanol, bixafen, blasticidin-S, boscalid, bromuconazole, bupirimate, buthiobate, carboxin, carpropamid, captafol, captan, carbendazim, chloroneb, chlorothalonil, chlozolinate, copper hydroxide, copper oxychloride, copper sulfate, coumoxystrobin, cyazofamid, cyflufenamid, cymoxanil, cyproconazole, cyprodinil, dichlobentiazox, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dipymetitrone, dithianon, dithiolanes, dodemorph, dodine, econazole, etaconazole, edifenphos, enoxastrobin (also known as enestroburin), epoxiconazole, ethaboxam, ethirimol, etridiazole, famoxadone, fenamidone, fenaminstrobin, fenarimol, fenbuconazole, fenfuram, fenhexamide, fenoxanil, fenpiclonil, fenpicoxamid, fenpropidin, fenpropimorph, fenpyrazamine, fentin acetate, fentin hydroxide, ferbam, ferimzone, flometoquin, florylpicoxamid, fluopimomide, fluazinam, fludioxonil, flufenoxystrobin, fluindapyr, flumorph, fluopicolide, fluopyram, fluoxapiprolin, fluoxastrobin, fluquinconazole, flusilazole, flusulfamide, flutianil, flutolanil, flutriafol, fluxapyroxad, folpet, fthalide (also known as phthalide), fuberidazole, furalaxyl, furametpyr, hexaconazole, hymexazole, guazatine, imazalil, imibenconazole, iminoctadine albesilate, iminoctadine triacetate, inpyrfluxam, iodicarb, ipconazole, ipfentrifluconazole, ipflufenoquin, isofetamid, iprobenfos, iprodione, iprovalicarb, isoflucypram, isoprothiolane, isopyrazam, isotianil, kasugamycin, kresoxim-methyl, lancotrione, mancozeb, mandipropamid, mandestrobin, maneb, mapanipyrin, mefentrifluconazole, mepronil, meptyldinocap, metalaxyl (including metalaxyl-M/mefenoxam), metconazole, methasulfocarb, metiram, metominostrobin, metyltetraprole, metrafenone, myclobutanil, naftitine, neo-asozin (ferric methanearsonate), nuarimol, octhilinone, ofurace, orysastrobin, oxadixyl, oxathiapiprolin, oxolinic acid, oxpoconazole, oxycarboxin, oxytetracycline, penconazole, pencycuron, penflufen, penthiopyrad, perfurazoate, phosphorous acid (including salts thereof, e.g., fosetyl-aluminm), picoxystrobin, piperalin, polyoxin, probenazole, prochloraz, procymidone, propamocarb, propiconazole, propineb, proquinazid, prothiocarb, prothioconazole, pydiflumetofen (Adepidyn®), pyraclostrobin, pyrametostrobin, pyrapropoyne, pyraoxystrobin, pyraziflumid, pyrazophos, pyribencarb, pyributacarb, pyridachlometyl, pyrifenox, pyriofenone, perisoxazole, pyrimethanil, pyrifenox, pyrrolnitrin, pyroquilon, quinaminoprole, quinconazole, quinmethionate, quinofumelin, quinoxyfen, quintozene, silthiofam, sedaxane, simeconazole, spiroxamine, streptomycin, sulfur, tebuconazole, tebufloquin, teclofthalam, tecloftalam, tecnazene, terbinafine, tetraconazole, thiabendazole, thifluzamide, thiophanate, thiophanate-methyl, thiram, tiadinil, tolclofos-methyl, tolprocarb, tolyfluanid, triadimefon, triadimenol, triarimol, triazoxide, tribasic copper sulfate, triclopyricarb, tridemorph, trifloxystrobin, triflumizole, trimoprhamide tricyclazole, trifloxystrobin, triforine, triticonazole, uniconazole, validamycin, valifenalate (also known as valifenal), vinclozolin, zineb, ziram, zoxamide, 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, and combinations thereof.

The pre-polymer compounds of the present invention can also be used to microencapsulate nematocides. Examples of nematocides include fluopyram, spirotetramat, thiodicarb, fosthiazate, abamectin, iprodione, fluensulfone, dimethyl disulfide, tioxazafen, 1,3-dichloropropene (1,3-D), metam (sodium and potassium), dazomet, chloropicrin, fenamiphos, ethoprophos, cadusaphos, terbufos, imicyafos, oxamyl, carbofuran, tioxazafen, Bacillus firmus, Pasteuria nishizawae, and combinations thereof. An example of a bactericide is streptomycin. Examples of acaricides include amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben, tebufenpyrad, and combinations thereof.

Of note is a composition comprising a compound of the invention (in an agriculturally effective amount), at least one additional active ingredient selected from the group consisting of other herbicides and herbicide safeners (in an effective amount), and at least one component selected from the group consisting of surfactants, solid diluents and liquid diluents.

The following abbreviations are used in the Index Tables which follow: The abbreviation “Cmpd. No.” stands for “Compound Number” or “Compound No.”. The abbreviation “Ex.” stands for “Example” and is followed by a number indicating in which Synthesis Example example the compound is prepared. Mass spectra are reported with an estimated precision within ±0.5 Da as the molecular weight of the highest isotopic abundance parent ion (M+1) formed by addition of H⁺ (molecular weight of 1) to the molecule observed by using atmospheric pressure chemical ionization (AP+).

### Index Table A

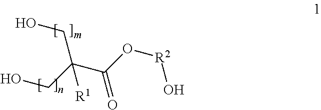

Starter isocyanate=m-XDI;

| | | | | | Mass |
|---|---|---|---|---|---|
| Cmpd. No. | m | n | R¹ | R² | (M + 1) |
| 1 (Ex. 1) | 1 | 1 | CH₃ | —CH₂CH₂CH₂— | 756 |

### EXAMPLES OF THE INVENTION

### Example A: Preparation of a Blank Polymer

A blank microencapsulation formulation was prepared using the reaction product of Compound No. 1 (see Index Table A) and m-xylylene diisocyanate (m-XDI: starter isocyanate). To prepare the blank microencapsulation polymer, the reaction product was dissolved in toluene and heated to 70 °C. This constituted the organic phase. Water, Reax 88B, Xiameter AFE100, Mowiol 8-88 (10% aqueous solution) and Chitosan (0.5% aqueous solution) were mixed and heated to 70 °C. The organic phase was added to the aqueous phase and emulsified. Upon adding the 1,6-hexamethylenediamine (di-amine) solution in the aqueous phase, an instantaneous reaction occurred at the organic droplet interface between the reaction product and 1,6-hexamethylenediamine, leading to stable capsule wall formation. The blank microcapsule slurry was centrifuged to wash out any components of the aqueous phase, then optionally washed with a water-immiscible solvent. Then the remaining polymer microcapsule was filtered, dried and collected.

TABLE 1
Blank formulation with Compound Number 1

| | Amount |
|---|---|
| Ingredients | (wt. %) |
| Toluene | 30.0 |
| Reax 88B | 1.50 |
| Mowiol 8-88 | 6.00 |
| (10% aqueous solution) | |
| Xiameter AFE 100 | 0.06 |
| Compound No. 1 | 4.80 |
| 1,6-hexamethylenediamine | 2.57 |
| (43% aqueous solution) | |
| Chitosan | 4.80 |
| (0.5% aqueous solution) | |
| Water | 50.27 |
| Total | 100.0 |

What is claimed is:

1. The reaction product of a tri-ol compound of Formula 1, stereoisomers thereof, 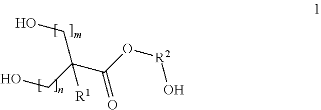
wherein
m is an integer from 1 to 4;
n is an integer from 1 to 4;
R¹ is selected from H, unsubstituted phenyl and linear and branched C₁-C₆ alkyl;
R² is selected from linear and branched C₂-C₆ alkyl and saturated or unsaturated C₅-C₆ cycloalkyl, optionally substituted with a linear or branched C₁-C₆ alkyl and at least one starter di-isocyanate compound.

2. The reaction product of the tri-ol compound of Formula 1 in claim 1 wherein
m is 1 to 3;
n is 1 to 3;
R¹ is selected from H and linear C₁-C₆ alkyl; and
R² is selected from linear and branched C₂-C₆ alkyl and saturated or unsaturated C₅ cycloalkyl, optionally substituted with a linear C₁-C₆ alkyl.

3. The reaction product of the tri-ol compound of Formula 1 in claim 2 wherein
m is 1 to 2;
n is 1 to 2;
R¹ is selected from H, —CH₃, —CH₂CH₃ and —CH₂CH₂CH₃; and
R² is selected from linear C₂-C₆ alkyl and saturated C₅ cycloalkyl, optionally substituted with a linear C₁-C₃ alkyl.

4. The reaction product of the tri-ol compound of Formula 1 in claim 3 wherein
m is 1;
n is 1;
R¹ is selected from H, —CH₃ and —CH₂CH₃; and
R² is selected from linear C₂-C₄ alkyl and saturated C₅ cycloalkyl, optionally substituted with CH₃.

5. The reaction product of the tri-ol compound of Formula 1 as defined in claim 1 wherein the at least one starter di-isocyanate compound is aliphatic, cycloaliphatic, or aromatic.

6. The reaction product of claim 5 wherein the at least one starter di-isocyanate compound is selected from 1,6-hexamethylene diisocyanate (HDI), 2,2,4- and 2,4,4-trimethyl-1,6-hexamethylene diisocyanate (TMDI), 1-isocyanato-3,3,5-trimethyl-5-isocyanatomethylcyclohexane (isophorone diisocyanate or IPDI), perhydro-2,4′- and -4,4′-diphenylmethane diisocyanate (HMDI), 1,4-diisocyanato-2,2,6-trimethylcyclohexane (TMCDI), 1,3- and 1,4-bis(isocyanato methyl)cyclohexane, m-xylylene diisocyanate (m-XDI), p-xylylene diisocyanate (p-XDI), m- and p-tetramethyl-1,3- and -1,4-xylylene diisocyanate (m- and p-TMXDI), 2,4- and 2,6-tolylenediisocyanate (TDI), 4,4′-2,4′- and 2,2′-diphenylmethane diisocyanate (MDI), polymeric MDI (PMDI), homologues thereof, isomers thereof, and mixtures thereof.

7. A polymer microcapsule prepared from the reaction product of claim 1 and an amine selected from a di-amine or a tri-amine.

8. The polymer microcapsule of claim 7 wherein the di-amine is selected from the group consisting of ethylenediamine (EDA), 1,2-propanediamine, 1,3-propanediamine (DAP), 2-methyl-1,2-propanediamine, 2,2-dimethyl-1,3-propanediamine, 1,3-butanediamine, 1,4-butanediamine, 1,3-pentanediamine, 1,5-pentanediamine, 2-butyl-2-ethyl-1,5-pentanediamine, 1,6-hexamethylenediamine (HMDA), methylbis(3-aminopropyl)amine, 1,5-diamino-2-methylpentane (MPMD), 1,2-diaminocyclohexane, 1,3-diaminocyclohexane, 1,4-diaminocyclohexane, bis(4-aminocyclohexyl)methane, bis(4-amino-3-methylcyclohexyl)methane, 1-amino-3-aminomethyl-3,5,5-trimethylcyclohexane (isophoronediamine or IPDA), m-xylylenediamine (XDA), 1,3-bis(aminomethyl)cyclohexane (BAC), methylene-bis(cyclohexylamine) (MBCHA), 4,4′-methylenebis(2-methylcyclohexylamine) (MBMCHA), homologues thereof, isomers thereof, and combinations thereof.

9. The polymer microcapsule of claim 8 wherein the di-amine compound comprises 1,6-hexamethylenediamine (HMDA).

10. The polymer microcapsule of claim 7 wherein the polymer is prepared by the process comprising:
a) forming a mixture comprising:
at least one reaction product of a trio-ol compound of Formula 1 as described in claim 1; and
at least one di-amine or tri-amine compound;
b) reacting the at least one reaction product of a trio-ol compound of Formula 1 and the at least one di-amine or tri-amine compound to form the polymer reaction product of the at least one reaction product of a trio-ol compound of Formula 1 and the at least one di-amine or tri-amine compound in the presence of an agrochemical compound; and
c) microencapsulating the agrochemical compound with the reaction product.

11. The reaction product as described in claim 1 that is [chemical structure]
